(19) United States
(12) Patent Application Publication
(10) Pub. No.: US 2009/0232698 A1
(43) Pub. Date: Sep. 17, 2009

(54) DEODORIZATION MATERIAL COMPOSITION, MANUFACTURING METHOD THEREOF, AND DEODORIZATION METHOD

(76) Inventor: Fei-Peng Lin, Hsinchu City (TW)

Correspondence Address: SINORICA, LLC, 2275 Research Blvd., Suite 500, ROCKVILLE, MD, 20850, US

(21) Appl. No.: 12/110,290

(22) Filed: Apr. 26, 2008

(30) Foreign Application Priority Data
Mar. 13, 2008 (TW) 097108809

(51) Int. Cl. A61L 9/00

(52) U.S. Cl. 422/5; 424/76.1

(57) ABSTRACT

A deodorization material composition, a manufacturing method thereof, and a deodorization method are provided. The deodorization material composition is adapted to eliminate an odor in an environment. The manufacturing method comprises providing nanoscale silver particles, nanoscale calcium particles, a nearly vacuum environment, a preset specific temperature, a medium material, and a post-treatment process, which allows the nanoscale silver particles and the nanoscale calcium particles to be alternately distributed in the medium material and thereby form a stable deodorization material composition. When the deodorization material composition is pressure sprayed into the environment, the nanoscale silver particles and the nanoscale calcium particles collide with or brush air particles to form free positive ions and negative ions respectively, which in turn react with constituents of the odor in the environment to eliminate the odor.

### BACKGROUND OF THE INVENTION

1. Field of the Invention

The present invention relates to a method for manufacturing a material composition and application thereof, and more particularly, to a method for eliminating an odor in an environment and a method for manufacturing a material composition used therewith.

2. Description of Related Art

Environmental fragrances, air fresheners, and deodorants abound in the market. However, the aforesaid products merely temporarily remove environmental odors and, more badly, are harmful to health due to their chemical constituents. The aforesaid products do not inhibit bacterial growth, and thus rotten substances with ongoing fermentation still produce odors of, for example, ammonia, alcohol, and phenol, in the presence of the aforesaid products. Hence, the aforesaid products fail to eradicate odors effectively. On the other hand, electronic products capable of inhibiting bacterial growth and effectively eradicating odors, such as air purifiers and air disinfection equipment, are neither available all the time nor portable. Therefore, a deodorization material composition, a manufacturing method thereof, and a deodorization method provided according to the present invention are intended to solve the aforesaid drawbacks of the prior art.

### SUMMARY OF THE INVENTION

To solve the aforesaid drawbacks of the prior art, a deodorization material composition, a manufacturing method thereof, and a deodorization method are provided according to the present invention, wherein the deodorization material composition eliminates an odor in an environment. The manufacturing method of deodorization material composition comprises steps of providing nanoscale silver particles, nanoscale calcium particles, a nearly vacuum environment, a preset specific temperature, a medium material, and a post-treatment process, which allows the nanoscale silver particles and the nanoscale calcium particles to be alternately distributed in the medium material and thereby form a stable deodorization material composition. When the material composition is pressure sprayed into the environment, the nanoscale silver particles and the nanoscale calcium particles collide with or brush air particles to form free positive ions and negative ions respectively, which in turn react with constituents of the odor in the environment to eliminate the odor.

Therefore, it is a primary objective of the present invention to provide a method for manufacturing a deodorization material composition capable of eliminating an odor and inhibiting bacterial growth, with a view to effectively eradicating odors of, for example, ammonia, alcohol, and phenol, which are produced as a result of fermentation of rotten substances.

Another objective of the present invention is to provide a method for manufacturing a deodorization material composition which has a high adsorption capacity and a high binding capacity so as to flocculate dust and mold in air, cleanse ambient air, and eradicate an odor instantly.

Yet another objective of the present invention is to provide a deodorization method for eliminating an odor and inhibiting bacterial growth, so as to effectively eradicate odors of, for example, ammonia, alcohol, and phenol, which are produced as a result of fermentation of rotten substances.

A further objective of the present invention is to provide a deodorization method, which uses a material composition having a high adsorption capacity and a high binding capacity so as to flocculate dust and mold in air, cleanse ambient air, and eradicate an odor instantly.

### BRIEF DESCRIPTION OF DRAWINGS

FIG. 1 is a flow chart illustrating a method for manufacturing a deodorization material composition according to a first preferred embodiment of the present invention; and

FIG. 2 is a flow chart illustrating a deodorization method according to a second preferred embodiment of the present invention.

### DETAILED DESCRIPTION OF PREFERRED EMBODIMENTS

A deodorization material composition, a manufacturing method thereof, and a deodorization method are disclosed in this specification, wherein the basic structures and properties of substances involved should be apprehensible to those ordinarily skilled in the art. Hence, a detailed description of such structures and properties will not be given herein. In addition, the appended drawings are intended to schematically illustrate features of the present invention and are therefore not, and need not be, drawn to scale.

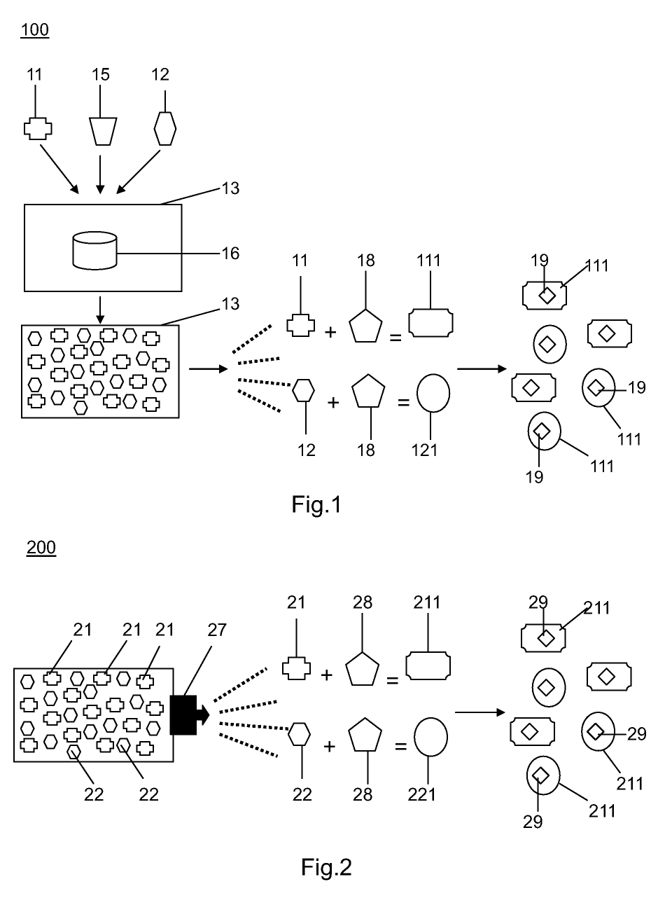

Refer to FIG. 1, which is a flow chart illustrating a method 100 for manufacturing a deodorization material composition according to a first preferred embodiment of the present invention, wherein the deodorization material composition is used to eliminate an odor in an environment. The method 100 for manufacturing the deodorization material composition comprises steps of:

(1) providing nanoscale silver particles 11;

(2) providing nanoscale calcium particles 12;

(3) providing a nearly vacuum environment 13;

(4) providing a preset specific temperature;

(5) providing a medium material 15; and

(6) providing a post-treatment process 16.

In the first preferred embodiment of the present invention, the nanoscale silver particles 11 and the nanoscale calcium particles 12 are put in the nearly vacuum environment 13 such that the nanoscale silver particles 11 and the nanoscale calcium particles 12 do not spread in air but stick closely together. The nearly vacuum environment 13 is preset to the specific temperature, preferably a low temperature, so that activities of the nanoscale silver particles 11 and the nanoscale calcium particles 12 are minimized. Afterward, the medium material 15 is provided, and then the post-treatment process 16 is performed, to allow the nanoscale silver particles 11 and the nanoscale calcium particles 12 to be uniformly and alternately distributed in between molecules of the medium material 15. The medium material 15 is preferably pure water while the post-treatment process 16 preferably involves vibrating, machine stirring, or manual stirring. With water molecules being larger than the nanoscale silver particles 11 and the nanoscale calcium particles 12, the post-treatment process 16 enables the nanoscale silver particles 11 and the nanoscale calcium particles 12 to be uniformly and alternately distributed in the pure water and form a stable material composition due to potential energy equilibrium. The stable material composition can be held in a container for preservation or transport because stability of the material composition remains unchanged even when the temperature rises or the material composition is not placed in a vacuum condition.

To remove the odor, the material composition is pressure sprayed, preferably at a pressure greater than an atmospheric pressure, into the environment. (For example, the material composition can be stored in a sprayer and sprayed out of the sprayer when necessary.) Due to a pressure difference and a diluting effect of the air, the nanoscale silver particles 11 and the nanoscale calcium particles 12 will rapidly collide with or brush air particles 18 and form free positive ions 111 and negative ions 121 respectively, which in turn react with constituents 19 of the odor in the environment. During the reaction, the positive ions 111 effectively eliminate the odor originating from fermentation of a rotten substance, while the negative ions 121, which are characterized by a high adsorption capacity and a high binding capacity, flocculate dust and mold in the air, cleanse ambient air, and eradicate the odor instantly.

As regards the manufacturing method described above, the preset specific temperature is preferably a low temperature because both the nanoscale silver particles 11 and the nanoscale calcium particles 12 have a low activity and low electrical potential energy at low temperatures, thereby allowing the stable material composition to be formed. The low temperature preferably ranges between 0° C. and 20° C., and more preferably between 5° C. and 10° C.

A major constituent of the nanoscale calcium particles 12 is powder ground from mineral stones having a degree of hardness of 8 or more, such as white Tourmaline and jade. Impurities in the mineral stone powder are charred in a plasma electrothermal process of above 1800° C. and then removed with a view to enhancing calcium purity. Afterwards, the mineral stone powder is ground again until nanoparticles are formed. In a preferred scenario, an ionization process is performed on the calcium particles to produce ionized calcium in order to enhance elimination of the odor.

As regards the manufacturing method described above, the step of providing the medium material 15 can be preceded by a pretreatment process, whereby the nanoscale silver particles 11 and the nanoscale calcium particles 12 are mixed into a mixture by stirring. In that case, the post-treatment process only involves pouring the medium material 15 into the mixture to disperse the nanoscale silver particles 11 and the nanoscale calcium particles 12 in the mixture and thereby ensures a good mix. Additional stirring or vibration further improves the mix.

The manufacturing method described above can further comprise a step of providing a preservation process (not shown), whereby the deodorization material composition for eliminating the odor is preserved in a container, such as a glass jar and a plastic bottle, at a room temperature and an atmospheric pressure, so that the deodorization material composition can be disposed as appropriate to be available at any time and portable.

Refer to FIG. 2, which is a flow chart illustrating a deodorization method 200 according to a second preferred embodiment of the present invention, wherein the deodorization method 200 comprises steps of:

(1) providing a material composition comprising nanoscale silver particles 21 and nanoscale calcium particles 22 mixedly disposed in a medium material;

(2) providing a pressure device 27; and

(3) using the pressure device 27 to spray the material composition into an environment, such that the nanoscale silver particles 21 and the nanoscale calcium particles 22 in the material composition collide with or brush air particles 28 in the environment to form free positive ions 211 and negative ions 221 respectively, which in turn react with constituents 29 of an odor in the environment to eliminating the odor.

The environment can be an air environment or alternatively comprise air and an object. Hence, the deodorization method 200 can be used to eliminate not only the odor in the air, but also an odor of the object.

The pressure device 27 can further comprise a nozzle device (not shown), such as a spray gun available on the market, for spraying the material composition into the environment. Other features of the material composition for use with the deodorization method 200 are the same as those disclosed in the first preferred embodiment.

The present invention has been described with preferred embodiments thereof for illustrative purposes only. The aforesaid embodiments should not be construed as to limit the scope of the present invention in any way. In addition, it is understood that the content disclosed herein should be readily understood and can be carried out by a person skilled in the art. Therefore, all equivalent changes and modifications which do not depart from the spirit of the present invention should be encompassed by the appended claims.

What is claimed is:

1. A method for manufacturing a deodorization material composition which is used to eliminate an odor in an environment, comprising steps of:
providing nanoscale silver particles;
providing nanoscale calcium particles;
providing a nearly vacuum environment;
providing a preset specific temperature;
providing a medium material; and
providing a post-treatment process to make the nanoscale silver particles and the nanoscale calcium particles be alternately distributed in the medium material, thereby forming a stable deodorization material composition;
wherein, when the material composition is pressure sprayed into the environment, the nanoscale silver particles and the nanoscale calcium particles collide with or brush air particles to form free positive ions and negative ions respectively, which react with constituents of the odor in the environment to eliminate the odor.

2. The method for manufacturing the deodorization material composition of claim 1, wherein the nanoscale calcium particles are ionized.

3. The method for manufacturing the deodorization material composition of claim 1, wherein the nanoscale calcium particles have been treated by a plasma electrothermal process to remove impurities thereof, in which the plasma electrothermal process is performed at an operating temperature higher than 1800° C.

4. The method for manufacturing the deodorization material composition of claim 1, further comprising a step of providing a pretreatment process before the step of providing the medium material, allowing the nanoscale silver particles and the nanoscale calcium particles to be mixed with each other in advance by the pretreatment process.

5. The method for manufacturing the deodorization material composition of claim 4, wherein the pretreatment process is selected from the group consisting of stirring, vibrating and the combination.

6. The method for manufacturing the deodorization material composition of claim 1, wherein the specific temperature is preset to a range of 0° C. to 20° C.

7. The method for manufacturing the deodorization material composition of claim 1, wherein the medium material is composed mainly of pure water.

8. The method for manufacturing the deodorization material composition of claim 1, wherein the material composition is pressure sprayed into the environment at a pressure greater than an atmospheric pressure.

9. The method for manufacturing the deodorization material composition of claim 1, wherein the post-treatment process comprises stirring, by which the medium material is disposed between the nanoscale silver particles and the nanoscale calcium particles with an appropriate pressure.

10. The method for manufacturing the deodorization material composition of claim 1, further comprising a step of providing a preservation process for preserving the deodorization material composition in a container at a room temperature and an atmospheric pressure.

11. A deodorization method for eliminating an odor in an environment, comprising steps of:
providing a material composition comprising nanoscale silver particles and nanoscale calcium particles mixedly disposed in a medium material;
providing a pressure device; and
using the pressure device to spray the material composition into the environment;
whereby the nanoscale silver particles and the nanoscale calcium particles in the material composition collide with or brush air particles in the environment to form free positive ions and negative ions respectively, which react with constituents of the odor in the environment to eliminate the odor.

12. The deodorization method of claim 11, wherein the nanoscale calcium particles are ionized.

13. The deodorization method of claim 11, further comprising a step of providing a container for storing the material composition.

14. The deodorization method of claim 11, wherein the pressure device further comprises a nozzle device.

15. The deodorization method of claim 11, wherein the medium material is composed mainly of pure water.

16. The deodorization method of claim 11, wherein the environment is an air environment.

17. The deodorization method of claim 11, wherein the environment comprises air and an object.

18. A deodorization material composition essentially comprising silver constituents and calcium constituents, characterized by:
the silver constituents being nanoscale silver particles; the calcium constituents being ionized nanoscale calcium particles; and further comprising a medium material which is composed mainly of pure water, so that the nanoscale silver particles and the ionized nanoscale calcium particles are uniformly and alternately distributed in the medium material.
